(19) United States
(12) Patent Application Publication
(10) Pub. No.: US 2005/0228295 A1
(43) Pub. Date: Oct. 13, 2005

(54) Method and system for dual domain discrimination of vulnerable plaque

(75) Inventor: Huwei Tan, Needham, MA (US)

Correspondence Address: HOUSTON ELISEEVA, 4 MILITIA DRIVE, SUITE 4, LEXINGTON, MA, 02421, US

(73) Assignee: InfraReDx, Inc., Cambridge, MA (US)

(21) Appl. No.: 10/816,110

(22) Filed: Apr. 1, 2004

(51) Int. Cl. A61B 5/02

(52) U.S. Cl. 600/481; 600/476; 600/478

(57) ABSTRACT

A method for optically analyzing blood vessel walls comprises receiving optical signals from the vessel walls and resolving a spectrum of optical signals in wavelength to generate spectral data. The spectral data is then transformed into the frequency domain. In the preferred embodiment, this transformation is achieved by applying wavelet decomposition. In other embodiments other transform techniques such as Fourier analysis is applied. The spectral data in the frequency domain are then used to analyze the vessel walls. In the typical embodiment, the spectral data are used to analyze a disease state of blood vessels walls such as the presence of atherosclerotic plaques, and their state. Dual domain method enables the spectral signals from blood vessels to be analyzed simultaneously according to frequency and wavelength (time). Dual-Domain Regression Analysis (DRDA) and Dual-Domain Discrimination Analysis (DDDA) in combination with wavelet transform (WT) enable the modeling of signals simultaneously in both domains. This provides a mechanism for isolating the non-interesting variation in spectra, making the system and analysis method more robust against variations in instrument and environmental conditions, e.g., broad-band spectral variation contributed from water, heart motion, and other non-interesting interferences. This provides higher sensitivity and specificity when compared with other models currently being used.

### BACKGROUND OF THE INVENTION

Chemometrics is the science of relating measurements made on a chemical system or process to the state of the system via application of mathematical and statistical methods. It is used many times to predict the properties, such as chemical composition, of structures based on their spectral response.

One application concerns the assessment of the state of blood vessel walls such as required in the diagnosis of atherosclerosis. This is an arterial disorder involving the intimae of medium- or large-sized arteries, including the aortic, carotid, coronary, and cerebral arteries. Atherosclerotic lesions or plaques can contain complex tissue matrices, including collagen, elastin, proteoglycans, and extracellular and intracellular lipids with foamy macrophages and smooth muscle cells. In addition, inflammatory cellular components (e.g., T lymphocytes, macrophages, and some basophiles) can also be found in these plaques.

Disruption or rupture of atherosclerotic plaques appears to be the major cause of heart attacks and strokes, because, after the plaques rupture, local obstructive thromboses form within the blood vessels.

Near infrared (NIR) spectroscopy can be used to measure and mathematical, including statistical, techniques applied to extract information from the NIR spectral data. Mathematical and statistical manipulations such as linear and non-linear regressions of the spectral band of interest and other multivariate analysis tools are available for building quantitative calibrations as well as qualitative models for discriminant analysis.

For example, in one specific spectroscopic application used in the identification of atherosclerotic lesions or plaques, an optical source, such as a tunable laser, is used to access or scan a spectral band of interest, such as a scan band in the near infrared of 750 nanometers (nm) to 2.5 micrometers (μm). The generated light is used to illuminate tissue in a target area in vivo using a catheter. Diffusely reflected light resulting from the illumination is then collected and transmitted to a detector system, where a spectral response is resolved. The response is used to assess the state of the tissue.

The environment in which the spectra are collected, however, creates problems. Due to the presence of intervening fluid, such as blood in the case of probes inserted into blood vessels, the spectral signals related to the properties of the tissue can be overwhelmed. Thus, robust discriminant methods must be used to extract the spectra of the vessel walls in the presence of noise sources. Further, the movement of the intervening fluid due to the heart's pumping action coupled with an inability to well control the probe head's distance from the region of interest on the blood vessel wall further work contrary to the precision required to enable accurate assessment of the vessel's state.

At a more macro level, the devices used to collect the spectra and natural variation between individuals provides added challenges. Discriminant methods must be robust against drift in the spectrometer and manufacturing differences between the, typically, disposable probes or catheters. The models based on the discriminant methods must be easily transferable and updatable and account for the drift and differences. Further, the discriminant methods must be able to compensate for nature individual-to-individual deviations in blood constituents and manifestations of the disease state.

### SUMMARY OF THE INVENTION

Spectra collected from most spectroscopic instruments are inherently local in nature owing to contributions from absorption, emission, the instrument, and measurement environment events occurring at different locations and with different localizations in both time (wavelength) domain and frequency.

Well-established algorithms based on direct application of regression by partial least squares (PLS) or principal component regression (PCR) are the most widely used methods for multivariate calculation. These algorithms globally explain spectral variance by using latent variables (or principal components) only in either the time (wavelength) or frequency domain, although separate variable selection by genetic algorithms or by other means can be used as a way of isolating localized effects in these modeling methods.

Without efficient isolation of localized effects, more global latent variables (or principal components) than necessary or desirable may have to be used to explain the local sources of variance in the time and frequency domains. As a consequence, the regression and discriminant models can be invalidated by the non-calibrated variation that is normally contributed from the fluctuation of sampling conditions. Significant baseline variation in near infrared (NIR) spectra, for example, can arise as a result of the heart's pumping action, intervening fluid, blood cell passing, blood distance variation, and catheter bending, all of which can degrade and even corrupt the discriminant analysis.

Mathematical transformations, the most widely-used one of which is the Fourier Transform (FT), translate signals from one domain to another domain. The FT, for example, transforms the NIR spectra that exist in the time domain (wavelength) to the frequency domain. Spectral features in wavelength domain are no longer local after the transformation, however. Instead, they are globally represented in frequency domain.

Wavelet transform (WT) is another form of mathematical transformation. It is similar to the traditional FT in that it takes a spectrum from a wavelength domain and represents it in the frequency domain. The WT, however, is distinguished from the FT by the fact that it not only dissects spectra into their frequency components in frequency domain, but it also varies the scale at which the frequency components are analyzed with a matched resolution. In other words, the WT allows spectra to be analyzed locally in both wavelength and frequency domains.

When applied to the spectral analysis of blood vessels, dual domain methods, such as WT, enable the spectral signals from blood vessels to be analyzed simultaneously according to frequency and wavelength. Specifically, Dual-Domain Regression Analysis (DDRA) and Dual-Domain Discrimination Analysis (DDDA) in combination with wavelet transform (WT) or other time-frequency transformation methods enable the modeling of signals simultaneously in both domains. This provides a mechanism for isolating and modeling the non-interesting variation in spectra, making the system and analysis method more robust against variations in instrument and environmental conditions, e.g., broad-band spectral variation contributed from water, heart motion, blood cell move, catheter bend variation, and other non-interesting interferences, while some other noises contributed from the laser speckle phenomenon in middle frequency range, due to constructive and destructive interference as using a tunable laser as the light source. This provides higher sensitivity and specificity, compared with other models currently being used.

Consequently, in general, according to one aspect, the invention features a method for optically analyzing blood vessel walls. The method comprises receiving optical signals from the vessel walls and resolving a spectrum of optical signals to generate spectral data.

In a typical implementation, the optical signal is tracked in time to obtain the spectrum. This is because the spectral response is usually obtained by detecting the response as a tunable source, illuminating the region of interest, is scanned over a spectral scan band or while a spectrometer analyzes the response of the region of interest, which is illuminated by a broadband source with array detectors. Alternatively FT-NIR systems can be used for spectrum acquisition.

According to the invention, the spectral data are partitioned into their frequency components in frequency domain. And the data are represented in both wavelength and frequency domains, which is defined as dual-domain spectra. The term “dual-domain” is used here because the spectra possess local features in both wavelength and frequency domains.

In the typical embodiment, this partition is achieved by applying the wavelet prism, which in one example involves the use of the Mallat pyramid algorithm for wavelet decomposition and application of the individual wavelet reconstruction afterwards. In other embodiments, other transform techniques and frequency filters, such as low-pass, high-pass, and band pass filter, can be applied to dissect the spectral information in the wavelength domain into dual-domain spectra. It is beneficial to note that those transform techniques should be designed to ensure that the dual-domain spectra are mutually orthogonal in Hilbert space. Ideally, the transformation process should be perfect or approximately perfect.

In any event, according to the invention, the dual-domain spectral data are then used to analyze the vessel walls. In the typical embodiment, the spectral data are used to analyze a disease state of blood vessels walls such as the presence of atherosclerotic plaques, and their state.

In some examples, dual domain regression analysis is used, such as with dual domain discrimination models. In some cases, the spectral data are preferably preprocessed before the dual domain transformation.

In other examples, regression analysis is used, such as with single domain discrimination models. However, in this example, the spectral data are preferably preprocessed by transforming the spectral data into dual-domain spectral data and then removing the undesired spectral variation by applying a signal correction operation to, such as low-frequency components of the dual-domain spectral data to reduce noise.

In general according to another aspect, the invention can also be characterized in the context of a system for optically analyzing blood vessel walls. This system comprises a detector system for receiving optical signals from the vessel walls and a spectrometer for resolving a spectrum of the optical signals in wavelength to generate spectral data. An analyzer then transforms the spectral data into dual-domain spectral data and uses the dual-domain spectral data to analyze the vessel walls.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a schematic diagram illustrating the application of a wavelet prism to the collected near infrared (NIR) spectra according to the present invention;

FIG. 2 is a schematic diagram illustrating the dual domain spectra, showing the absorption both as a function of frequency and wavelength, illustrating the expansion of the data into the frequency and wavelength domains according to the present invention;

FIG. 3 is a plot of a NIR spectra simulating the contribution of three factors, the signal of interest, baseline variation, and high frequency noise;

FIG. 4 is a plot of spectral variation as a function of wavelet scale illustrating the location of the analytical signal in the frequency domain;

FIG. 5A is a schematic block diagram illustrating the spectroscopic catheter system to which the present invention is applicable;

FIG. 5B is a cross-sectional view of the catheter head positioned for performing spectroscopic analysis on a target region of a blood vessel;

FIG. 6 is a schematic block diagram illustrating the calibration step of a dual-domain Mahalanobis discriminator according to one embodiment of the present invention;

FIG. 7 is a schematic block diagram illustrating the prediction step of the dual-domain Mahalanobis discriminator;

FIG. 8 shows the application of the dual domain partial least squares discrimination algorithm to the dual domain data set to obtain the discrimination algorithm model according to the present invention;

FIG. 9 illustrates the application of the partial least squares dual domain discrimination algorithm according to one embodiment of the present invention;

FIG. 10 schematically illustrates the generated dual domain partial least squares discrimination analysis DDPLS-DA model according one embodiment of the present invention;

FIG. 11 is a plot of accuracy as a function of model factors showing the decreased number of model factors associated with the dual domain analysis of the present invention; and

FIG. 12 is a plot of mean sensitivity and specificity as a function of blood distance between the catheter head and the target area of the vessel wall, illustrating the insensitivity achieved by the present invention relative to this blood distance.

### DETAILED DESCRIPTION OF THE PREFERRED EMBODIMENTS

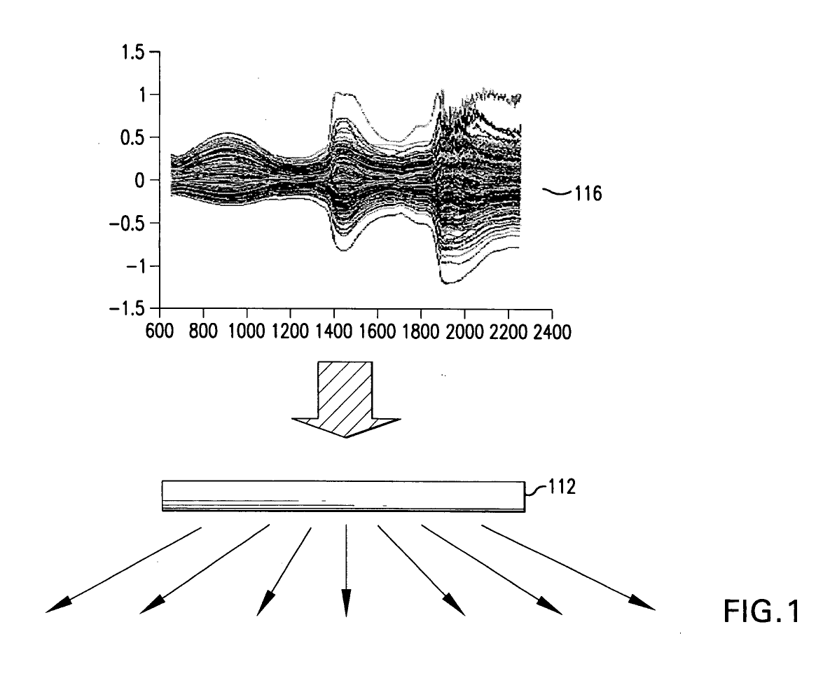

FIG. 1 illustrates the partitioning of spectral data that were acquired from a blood vessel.

Specifically, a set of near infrared (NIR) spectra are shown in the graph inset 116. In the current embodiment, these spectra were collected from a region, or regions, of interest on the interior of a patient's blood vessel, such as the coronary artery. Specifically, the plot shows mean-centered absorbance as a function of wavelength in nanometers (nm) covering a scan band of 600 to 2300 nm. In some implementations, the scan band is represented in time corresponding to the capture or resolving device's time to scan over the band of interest to collect each spectrum.

The spectra exhibit a large degree of variability between individual scans. Some of this variability is due to signals from the regions of interest. However, most of variability is due to the combined effects of noise sources in the time and frequency domains.

A wavelet prism algorithm 112 splits a time-domain spectra into a set of dual-domain spectra. In one example, an implementation of the Mallat pyramid algorithm coupled with wavelet reconstruction is used.

In some implementations some prefiltering or pre-scaling is applied to the spectral data prior transformation into the dual-domain space, such as mean centering. More generally, preprocessing is applied as described in U.S. patent application Ser. No. 10/426,750, filed on Apr. 30, 2003, entitled Spectroscopic Unwanted Signal Filters for Discrimination of Vulnerable Plaque and Method Therefor, by Marshik-Geurts, et al., this application being incorporated herein in its entirety by this reference.

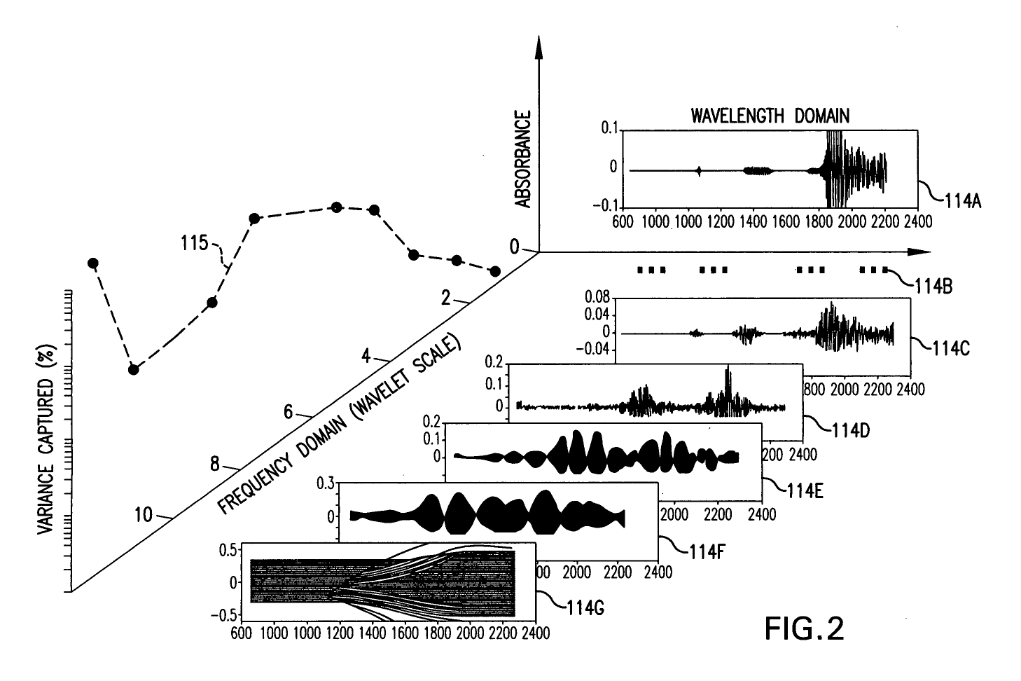

FIG. 2 shows a set of wavelet representations 114A-114G of the original data by action of the wavelet prism decomposition 112 on the original spectra.

Specifically, it illustrates the local nature of the transformed data. The data now show the absorption both as a function of wavelength and as a function of frequency in wavelet scales. The localized variation in the spectral data is expanded into the frequency domain. Specifically, each of the separate plots 114A-114G shows how the spectral data are distributed in two domains. The plot 115 illustrates the total distribution of the spectra over frequency domain.

This decomposition of the response matrix X for m samples measured at p spectral wavelengths, using a wavelet prism in the current embodiment, can be formulated as:

$$X \approx {\sum }_{k = 1}^{l + 1} {X}_{k} where \\ {X}_{1} = {G}^{T} {D}^{1} \\ {X}_{2} = {H}^{T} {G}^{T} {D}^{2} \\ … \\ {X}_{1} = \underbrace{{H}^{T} {H}^{T} … {H}^{T}}_{1 - 1} {G}^{T} {D}^{1} \\ {X}_{1 + 1} = \underbrace{{H}^{T} {H}^{T} … {H}^{T}}_{1} {A}^{1} \qquad ( 1 )$$

The decomposition at the wavelet scale (level) l yields a m×p×(l+1) dual-domain, spectral cubic X including l+1 frequency components {X₁, X₂, . . . , X₁, Xₗ₊₁}. The matrices D¹, D², . . . , D^{k}, . . . , D¹, and A obtained by wavelet decomposition using the Mallat algorithm denote the wavelet coefficients. H and G are a low-pass and a high-pass filter, respectively, and are determined by the specific mother wavelet used in the transform.

For the other methods of generating dual-domain spectra, the time-frequency transform and decomposition are implemented by optimizing a set of basis vectors with the available a priori knowledge about analytes of interest and interferants, to maximize the separation between the various sources.

In the current embodiment, the decomposition differs from that often used since there is no wavelength compression with increasing scale. This permits examination and selective removal of certain local features with restricted frequency characteristics.

As shown in FIG. 2, “baseline-like” aspects of the spectra (low-frequency components and noise), which are mainly related to the blood distance variation, heart motion, and catheter curvature difference, are more concentrated in the lowest-frequency approximation component 114G and comprise a majority, approximately 98%, of total spectral variance in many instances. The high-frequency noise, which may mostly result from the modal hopping of the laser light source, can be found in the low-scale representations 114A and 114B. These high frequency components comprise small spectral variance of the dual-domain spectra produced by the decomposition. They often contain little contribution from the spectral variation caused by the chemical or physical properties of interest when compared with the components in the frequency ranges that describe most typical spectral peaks.

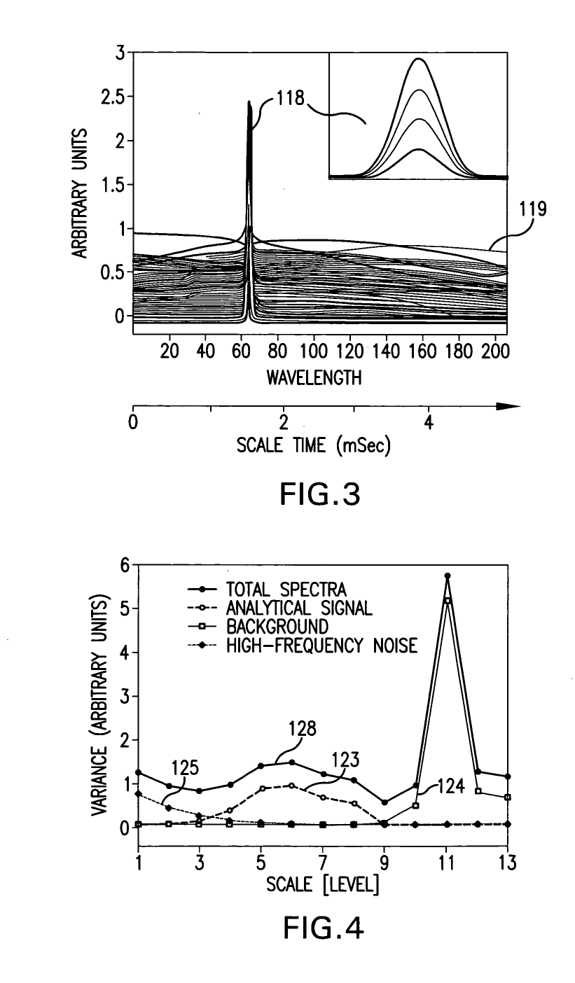

FIG. 3 shows a set of simulated spectra, which include the analytical signal (the graph insert 118), broad band baseline (the 119), and high-frequency noise. Each spectrum with more than 2000 wavelength points is collected in 5 milliseconds.

FIG. 4 is a plot of spectral variance of the simulated spectra as a function of wavelet scale that spans most of the frequency region. It illustrates the localization of various sources in the frequency domain.

Generally, the total spectra 128 (solid point) can be decomposed into three type of sources, signal 123 (dash and hollow point), high frequency noise 125 (dotted line and solid point), and baseline or low frequency noise 124 (dotted line hollow square).

Only the frequency domain has been shown here in FIG. 4. The x-axis is the wavelet scale, corresponding to frequency domain, from 1 (high frequency) to 13 (low). The y-axis is in arbitrary units, which indicates spectral variation.

A large value means large portion of spectral intensity contributed into the total spectra 128.

The baseline is located around 11 and higher levels on the wavelet scale, while high frequency noise has a significant contribution to the total spectra via the low frequency domain (1˜4 level). The signal of interest is mostly located in the middle range of frequencies. Therefore the signal of interest can be usually extracted by using frequency filtering techniques.

It should be noted, however, that simple spectral filtering will not match the performance of the dual domain approach. This is because, while the sources are localized in frequency domain, the noise is distributed over the whole frequency domain. That is to say, the noise contribution is not zero at the frequency location where signal is present. Thus, the frequency-based filters will also remove the signal of interest, which translates to lost information.

A linear transform such as the wavelet decomposition preferably conserves the relationship of property to spectra through the decomposition. Therefore, the frequency components in dual-domain spectra obtained by wavelet prism decomposition may be modeled separately at different frequency scales, if a linear relationship between the raw spectra and the target property exists. As a result, it is possible to implement a regression or discrimination analysis on the dual-domain spectra produced from a wavelet prism decomposition of a set of spectra over the entire wavelength and frequency domains at the same time, providing a way to isolate local information without significant information loss.

The dual-domain approach, however, will keep all of the spectral variation and do the processing in the model calibration step, which will decrease the chance of information loss and increase the chance of extracting the interesting information.

It is important to mention that, the dual-domain approach can also be used to do signal correction in preprocessing step, which will increase the chance of separating the interest information from the undesired variation.

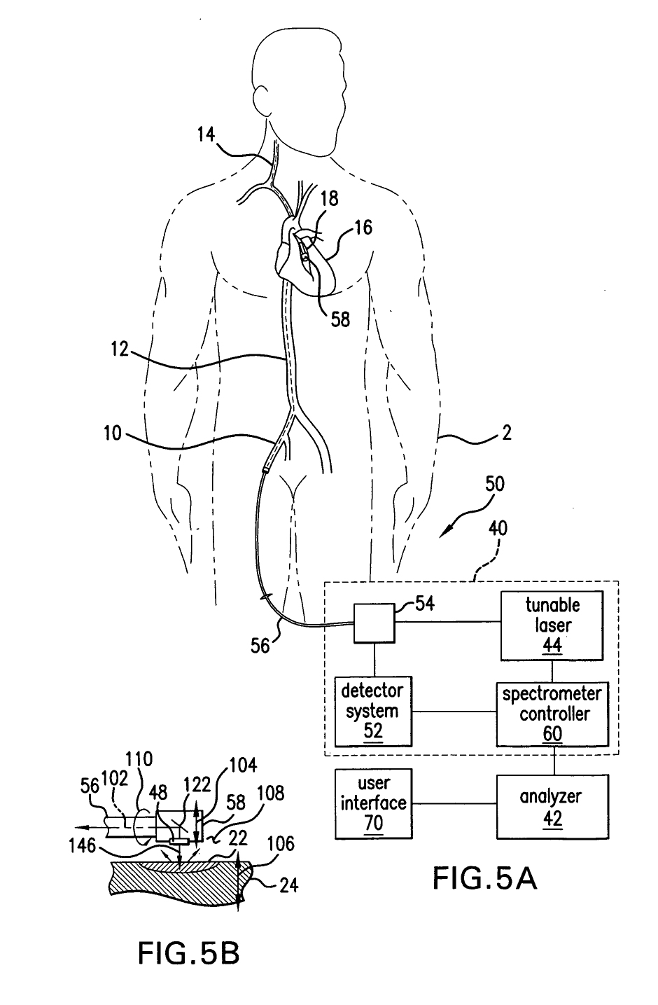

FIG. 5A shows an optical spectroscopic catheter system 50 for blood vessel analysis, to which the present invention is applicable, in one embodiment.

The system 50 generally comprises a probe, such as catheter 56, a spectrometer 40, and analyzer 42.

In more detail, the catheter 56 includes an optical fiber or optical fiber bundle. The catheter 56 is typically inserted into the patient 2 via a peripheral vessel, such as the femoral artery 10. The catheter head 58 is then moved to a desired target area, such as a coronary artery 18 of the heart 16 or the carotid artery 14. In the embodiment, this is achieved by moving the catheter head 58 up through the aorta 12.

When at the desired site, radiation is generated. In the current embodiment, optical illuminating radiation is generated, preferably by a tunable laser source 44 and tuned over a range covering one or more spectral bands of interest. In other embodiments, one or more broadband sources are used to access the spectral bands of interest. In either case, the optical signals are coupled into the optical fiber of the catheter 56 to be transmitted to the catheter head 58.

In the current embodiment, optical radiation in the near infrared (NIR) spectral regions is used. Exemplary scan bands include 1000 to 1450 nanometers (nm) generally, or 1000 nm to 1350 nm, 1150 nm to 1250 nm, 1175 nm to 1280 nm, and 1190 nm to 1250 nm, more specifically. Other exemplary scan bands include 1660 nm to 1740 nm, and 1630 nm to 1800 nm. In some implementations, the spectral response is first acquired for a full spectral region and then bands selected within the full spectral region for further analysis.

However, in other optical implementations, scan bands appropriate for fluorescence and/or Raman spectroscopy are used. In still other implementations, scan bands in the visible or ultraviolet regions are selected.

In the current embodiment, the returning, diffusely-reflected light is transmitted back down the optical fibers of the catheter 56 to a splitter or circulator 54 or in separate optical fibers. This provides the returning radiation or optical signals to a detector system 52, which can comprise one or multiple detectors.

A spectrometer controller 60 monitors the response of the detector system 52, while controlling the source or tunable laser 44 in order to probe the spectral response of a target area, typically on an inner wall of a blood vessel and through the intervening blood or other unwanted signal sources.

As a result, the spectrometer controller 60 is able to collect spectra by monitoring the time varying response of the detector system 52. When the acquisitions of the spectra are complete, the spectrometer controller 60 then provides the data to the analyzer 42.

With reference to FIG. 5B, the optical signal 146 from the optical fiber of the catheter 56 is directed by a fold mirror 122, for example, to exit from the catheter head 58 and impinge on the target area 22 of the artery wall 24. The catheter head 58 then collects the light that has been diffusely reflected or refracted (scattered) from the target area 22 and the intervening fluid 108 and returns the light 102 back down the catheter 56.

In one embodiment, the catheter head 58 spins as illustrated by arrow 110. This allows the catheter head 58 to scan a complete circumference of the vessel wall 24. In other embodiments, the catheter head 58 includes multiple emitter and detector windows, preferably being distributed around a circumference of the catheter head 58. In some further examples, the catheter head 58 is spun while being drawn-back through the length of the portion of the vessel being analyzed.

However the spectra are resolved from the returning optical signals 102, the analyzer 42, transforms the data to obtain the dual domain data set. From here, an assessment of the state of the blood vessel wall 24 or other tissue of interest is made from collected spectra. This assessment is made using, for example, Dual-Domain Regression Analysis (DDRA) and Dual-Domain Discrimination Analysis (DDDA), in some exemplary embodiments.

The collected spectral response is used to determine whether the region of interest 22 of the blood vessel wall 24 comprises a lipid pool or lipid-rich atheroma, a disrupted plaque, a vulnerable plaque or thin-cap fibroatheroma (TCFA), a fibrotic lesion, a calcific lesion, and/or normal tissue in the current application. In another example, the analyzer makes an assessment as to the level of medical risk associated with portions of the blood vessel, such as the degree to which portions of the vessels represent a risk of rupture. This categorized or even quantified information is provided to an operator via a user interface 70, or the raw discrimination or quantification results from the collected spectra are provided to the operator, who then makes the conclusion as to the state of the region of interest 22.

In one embodiment the information provided is in the form of a discrimination threshold that discriminates one classification group from all other spectral features. In another embodiment, the discrimination is between two or more classes from each other. In a further embodiment the information provided can be used to quantify the presence of one or more chemical constituents that comprises the spectral signatures of a normal or diseased blood vessel wall, or the vulnerability index that is defined as the measure of the risk of heart attack.

The dual domain analysis can be used to address the relative motion between the catheter head 58 and the vessel wall 24. Movement in the catheter head 58 is induced by heart and respiratory motion. Movement in the catheter head 58 is also induced by flow of the intervening fluid 108, typically blood. The periodic or pulse-like flow causes the catheter head 58 to vibrate or move as illustrated by arrow 104. Further, the vessel or lumen is also not mechanically static. There is motion, see arrow 106, in the vessel wall 24 adjacent to the catheter head 58. This motion derives from changes in the lumen as it expands and contracts through the cardiac cycle. Other motion could be induced by the rotation 110 of the catheter head 58. Thus, the relative distance between the optical window 48 of catheter head 58 and the region of interest 22 of the vessel 24 is dynamic.

Regression Analysis

The regression analysis on a dual-domain spectral set is a two-step procedure, done in a way similar to that used for regular (single-domain) regression methods. The first step is to establish a dual-domain model in a calibration set between the dependent m×1 vector y (the property) and a set of independent variables contained in a dual-domain spectral cubic X{Xₖ, k=1, 2, . . . , 1+1}. The second step is to predict values for the dependent properties based on a prediction set Xᵤ={X^{T}_{1,u} . . . X^{T}_{l+1,u}}^{T}.

Consider the dual-domain regression model

$$y = {\sum }_{k = 1}^{l + 1} {X}_{k} {β}_{k} + e E ( e ) = 0 , Cov ( e ) = {σ}^{2} I \qquad ( 2 )$$

where βₖ is the p×1 regression coefficient vector for the frequency component at the kth scale in the dual-domain spectra, e denotes an m×1 error vector, and E(·) and Cov(·) are the expectation and covariance, respectively. The goal of the dual-domain regression analysis is to calculate the regression coefficients β={β₁, . . . , βₗ₊₁} with the lowest associated prediction error. Principal Component Regression (PCR), Partial Least Squares (PLS), continuum regression (CR), ridge regression (RR), and regression with a maximum likelihood criterion or a Bayesian information criterion are common approaches useful for the regression step.

In dual-domain PCR (DDPCR), the regression vector is determined by

$${\hat{β}}_{DD PCR} = AGR {min}_{{β}_{DDPCR} ∈ R} [ {Σ ( y - \hat{y} )}^{2} ] \qquad ( 3 )$$

Exact solution of the equations (2) or (3) for the optimal model defined there is not straightforward. However, satisfactory performance may be obtained by an approximate solution for this model.

Consider dual-domain regression using PCR. To find an approximate solution to equation 3, several steps are involved. In this case, a separate PCR on each frequency component of the dual-domain spectra is first performed with respect to an analytical target, the dependent vector y, and the PCR regression vector obtained is then weighted according to the predictive ability of each frequency domain component for the target. The frequency component with highest linear relationship to the analytic target will gain the highest weight. Cross-validation methods are preferably employed here for the PCR models of frequency components to extract this frequency distribution.

The singular value decomposition (SVD) of the kth frequency component of the dual-domain spectra X, Xₖ, is expressed by Xₖ=UₖΣₖVₖ^{T}. The matrix Uₖ represents the m×qₖ matrix of eigenvectors for XₖXₖ^{T}, Vₖ symbolizes the p×qₖ matrix of eigenvectors for Xₖ^{T}Xₖ, and Σₖ denotes the qₖ×qₖ diagonal matrix of singular values (σ_{i,k}) equal to the square root of the eigenvalues of XₖXₖ^{T} and Xₖ^{T}Xₖ. Note that the rank, qₖ, of Xₖ will vary with scale. The PCR modeling approach is to include the first d eigenvectors (d≦qₖ) pertinent in modeling the prediction property, where d represents the prediction rank. A general form of the DDPCR regression vector \hat{β}_{k,DDPCR} for the kth frequency scale is expressed by

$${\hat{β}}_{k , DDPCR} = {g}_{k} [ {\sum }_{i = 1}^{d} ( {σ}_{i , k}^{- 1} {u}_{i , k}^{T} y ) {v}_{i , k} ] = {g}_{k} {\hat{β}}_{k , PCR} \qquad ( 4 )$$

where \hat{β}_{k, PCR} is separately estimated by regular PCR for the frequency component at the kth scale. The scalar term, gₖ, that is typically associated with the frequency distribution of the analytic target over frequency domain, is the weight for the kth scale determined by the receiver operating characteristic—area under curve (ROC-AUC) analysis or cross-validation (CV) of the calibration set (for medical diagnosis discrimination) according to

$$\begin{matrix}{g}_{k} = {AUC}_{k} / {\sum }_{k = 1}^{l + 1} {AUC}_{k} & ( 5 a ) \\ {g}_{k} = {s}_{k}^{2} / {\sum }_{k = 1}^{l + 1} {s}_{k}^{2} & ( 5 b ) \\ g = AGR {max}_{g ∈ R} ( FOM ) & ( 5 c )\end{matrix}$$

In equation 5a, AUCₖ denotes the area obtained from the receiver operating characteristics curve under area (ROC-AUC) analysis in the calibration set for kth scale, while sₖ in equation 5b is the reciprocal of the cross-validation error. In addition, this coefficient term, g (gₖ, k=1, 2, . . . , l+1), can be optimized by maximizing the value in Figure of merit (FOM), according to equation 5c. FOM is defined to measure the performance of predicting vulnerability for a risk of heart attack.

In the prediction step, an unknown sample x^{T}ᵤ is first decomposed by the WP algorithm, followed by multiplication of the frequency components x^{T}_{k,u}(k=1,2, . . . 1, 1+1) with the kth regression vector according to

$${\hat{y}}_{u} = {\sum }_{k = 1}^{l + 1} {x}_{k , u}^{T} {\hat{β}}_{k , DDPCR} \qquad ( 6 )$$

Similarly, for dual-domain regression using PLS (DDPLS), CR (DDCR), RR (DDRR), an approximate solution to equation (2) can be obtained as
\hat{β}_{k, DD-RGN}=gₖ \hat{β}_{k, RGN}, where RGN=PLS, CR, RR, (7)
where \hat{β}_{k, RGN} is computed separately by regular regression analysis on the kth-scale frequency component, and the weight gₖ for the kth scale is estimated by the ROC-AUC analysis, cross-validation of the calibration set, or optimization method.

It should be clear that because the weighting of the regression defined in equations (4) and (7) combines the sets of latent variables generated from the separate analyses of the wavelet decompositions at different scales, there will be only a single set of latent variables produced from DDRA, just as in regular regression analysis (e.g., PLS or PCR). However, the weighted latent variables produced by DDPCR and DDPLS, in general, will differ from those produced by conventional PCR and PLS, respectively, because of the weighting of the sets of latent variables. A performance comparison with those from PCR or PLS done in terms of latent variables from each method can be done to see if there is benefit to the dual domain analysis, even though the variables used in the comparison are not directly equivalent. Such a comparison is analogous to those done, for example, between PLS and PCR.

Discrimination Analysis

In another implementation, a multivariate regression technique is built distinguishing the differences between two classifications or other classification schemes of interest. In a current implementation, the regression technique used is PLS-DA. The PLS-DA model is based upon maximizing the separation of the information based upon the groups to be distinguished. A threshold is established by a classifier providing the mechanism for separating samples from all other groups or samples. The classifier can also provide the calculated results of the scores from the model.

In another embodiment, a calibration model based upon machine learning techniques is built distinguishing the differences between two classifications schemes, or more, of interest. The classification is provided by the application of the machine learning system approach that determines which combinations of the measurements are sufficient to distinguish between the classes. These methods can be applied as non-linear or linear separators. In one embodiment, artificial neural networks are used and the method is fine tuned by changing the number of degrees of freedom or dimensionality of the model. In another embodiment, support vector machines form hyper-planes between the assigned classes and in general attempt to maximize the separation between the two closest points in each classification group.

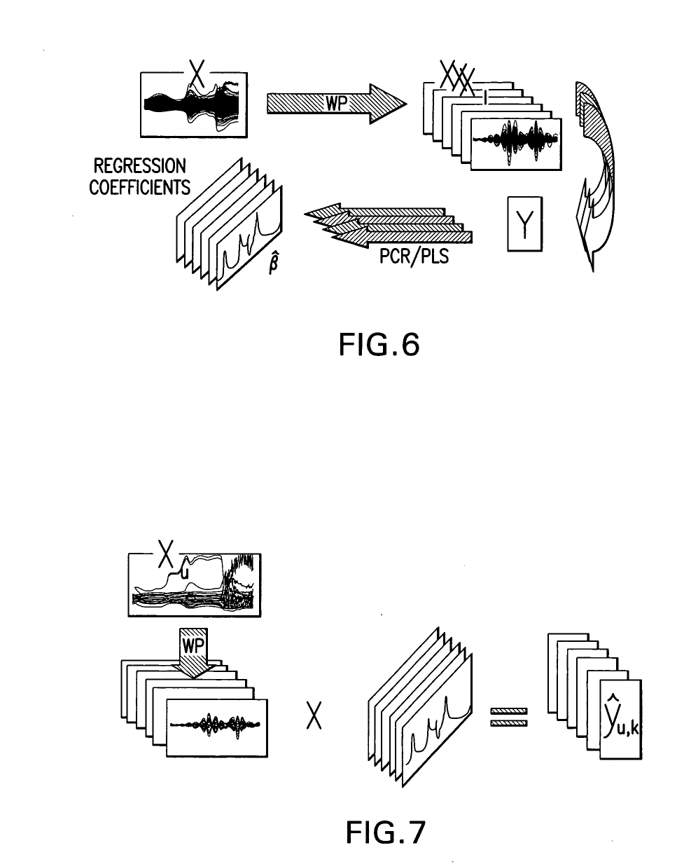

In a further, preferred, embodiment, Mahalanobis classifiers (discriminators) are used on the dual-domain spectra. As opposed to the weights strategy used in Equations 4, 5, and 7, the dual-domain Mahalanobis discriminators automatically account for the scale differences between frequency components. They provide a curved or linear boundary surface (threshold) in the high-dimension Hilbert space to improve the discrimination decision making. Basically in these methods, as shown in FIG. 6, a set of parallel multivariate regression models are established separately on the frequency components in dual-domain spectra, The estimation of sensitivity (positive, e.g., LP and DP) samples in calibration set, Ŷₚ, is used to compute the Mahalanobis distance (MD), according to
MD²=(Ŷₚ−m_{Ŷ}_{ₚ})′C_{Ŷ}_{ₚ}⁻¹(Ŷₚ−m_{Ŷ}_{ₚ}) (8)
where m_{Ŷ}_{ₚ} is the mean of Ŷₚ, and C_{Ŷ}_{ₚ} is the covariance matrix of Ŷₚ. The Mahalanobis distances of specificity samples (negative, e.g., Fibrotic (F13) and Calcific (CAL) are also calculated by using the covariance matrix C_{Ŷ}_{ₚ} and the estimation of specificity samples Ŷₙ. The ROC analysis is then conducted on both two groups' MDs to determine the discrimination threshold for the final dual-domain Mahalanobis discriminator.

As shown in FIG. 7, in the prediction step of unknown spectra Xᵤ, are passed through the wavelet prism (WP), the parallel models are applied to the partitioned spectra, leading to a set of prediction scores ŷ_{u,k}(k=1,2, . . . , l+1), following by calculation of Mahalanobis Distance.

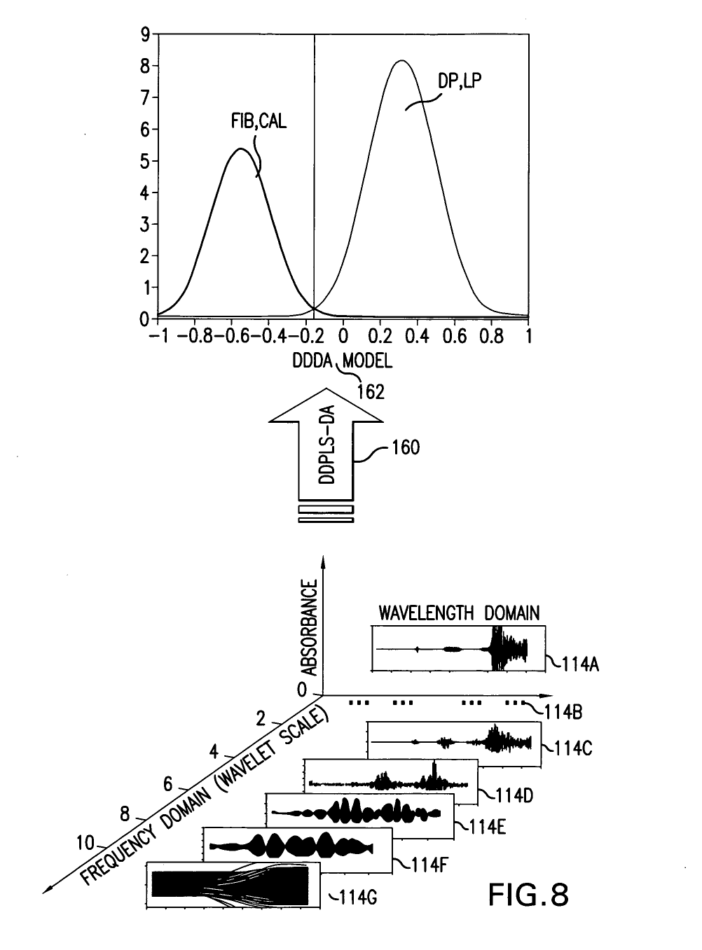

FIG. 8 shows the strategy used in the current embodiment. The dual domain (DD) PLS-DA algorithm 160 is applied to the dual domain transformed data sets 114A-114G. Spectra are then separated into two classification groups using the dual domain discrimination model 162. In current examples, one group is the Lipid Pool (LP) and Disrupted Plaque (DP) sample prediction results and the other is for Fibrotic (FIB) and Calcific (CAL) sample prediction, according to one classification scheme. In another embodiment, the scheme distinguishes between vulnerable plaques or thin-cap fribroatheroma (TCFA) and non-vulnerable plaques or non-TCFA.

The core of the PLS-DA algorithm for the dual domain analysis currently used is a spectral decomposition step performed via either the NIPALS or the SIMPLS algorithm.

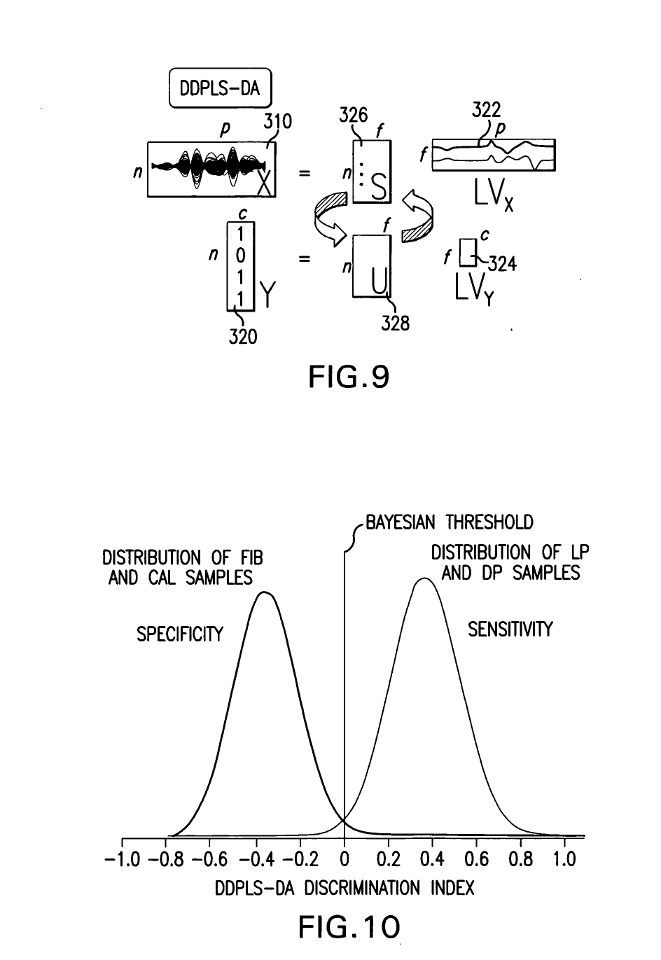

FIG. 9 is a diagram representing the NIPALS decomposition of the spectral information represented by the X matrix 310 and the binary classification information represented by the Y matrix 320.

X 310 is the spectra data matrix, Y 320 is the binary component information matrix, S and U are the resultant scores matrix 326, 328 from the spectral and component information respectively and LVx 322 and LVy 324 are the loading scores of latent variables (LV) for spectra and information, respectively. The other nomenclature is for the number of spectra (n), the number of data points (p), the number of components (c), and the number of final principal components (f).

Once the first decomposition is made resulting in a LV and scores for each of the X and Y matrices, the resultant scores matrix for the spectral information (S) 326 is swapped with the scores matrix containing the binary classification information (U) 328. The latent variable information from LVx and LVy 322, 324 are then subtracted from the X and Y matrices 310, 320, respectively. These newly reduced matrices are then used to calculate the next LV and score for each round until enough LVs are found to represent the data. Before each decomposition round, the new score matrices are swapped and the new LVs are removed from the reduced X and Y matrix.

The final number of latent variables arrived at from the PLS decomposition (see f) are highly correlated with the group classification information due to the swapped score matrices. The LVx and LVy matrices contain the highly correlated variation of the spectra with respect to the two groups used to build the model. The second set of matrices, S and U, contain the actual scores that represent the amount of each of the principle component variation that are present within each spectrum.

The scores from the U matrix and X-block weights are used to calculate the regression coefficients for each frequency components. According to Equations 7 and 5, the final dual-domain discrimination model is established, as represented in FIG. 10. The threshold was set using the model discrimination indices for the LP and DP scores as one group and those for the FIB and CAL as the other group according to one classification scheme for the blood vessels. For predictions, an unknown spectrum was dissected by wavelet prism, followed by a prediction according to Equation 6, leading to the DDPLS-DA discrimination index. If this resultant value is above the threshold of the model then that sample is said to be either a member of the LP and/or DP class.

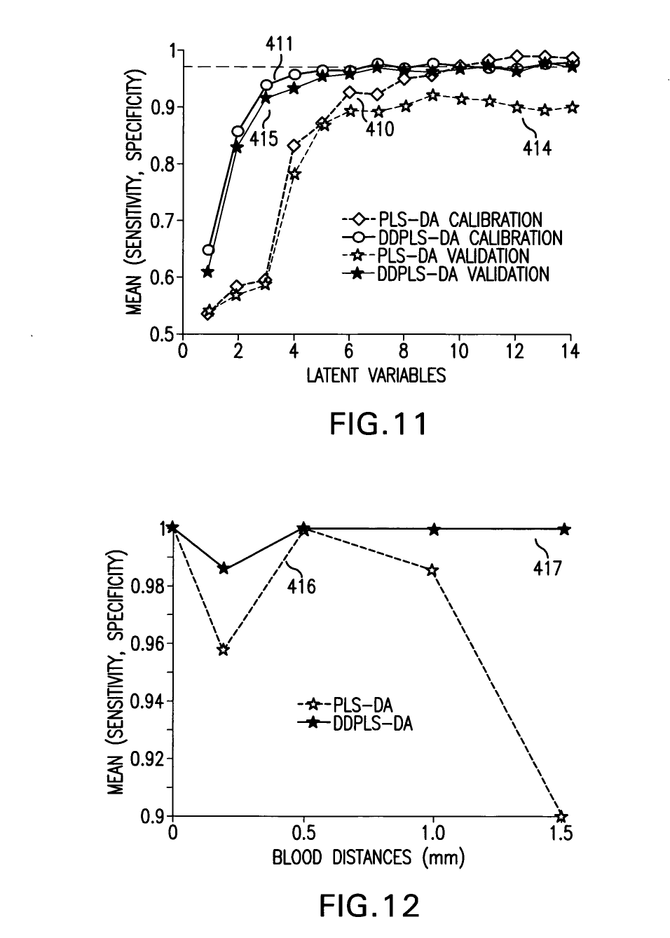

FIG. 11 illustrates the improved performance associated with the dual domain partial least squares discrimination analysis DDPLS-DA, as opposed to convention single domain PLS-DA algorithms. In the figure, x-axis is the latent variable number used in models, while y-axis presents the mean value of sensitivity of specificity, corresponding to the discrimination performance. Two curves, 410 and 411, are the cross-validation results for PLS-DA (dotted line and hollow square) and DDPLS-DA (solid and hollow circle), respectively. This suggests that DDPLS-DA needs fewer latent variables than the regular PLS-DA.

The other two curves, 414 and 415, show the results from the blind validation for both methods. The DDPLS-DA provided improved performance in terms of decreasing the LV number required and significantly enhancing the sensitivity and specificity. On other hand, the 411 and 415 from DDPLS-DA models almost overlap, while the 410 and 414 diverge when the latent variables is larger than 6. This implies that the regular PLS-DA models suffered from over-fitting and DDPLS-DA models performed consistently. Compared with regular PLS-DA, DDPLS-DA, therefore, is more robust and easier to maintain, update, or transfer, and is able to be applied to a broader number of situations.

In addition, FIG. 12 illustrates the mean sensitivity/specificity as a function of blood distance between the catheter head 58 and the target area 22. The plot, 417, shows the general insensitivity of the dual domain partial least squares discrimination algorithm to distances between 0 and 1.5 millimeters. In contrast, the conventional single domain PLS discrimination algorithm, as shown in plot 416, exhibits a sharp fall off from approximately 0.98 to 0.9 when distances in excess of 1 millimeter are encountered.

Dual Domain Preprocessing

Referring back to FIG. 1, a wavelet prism algorithm 112 splits a time-domain spectra into a set of dual-domain spectra. As shown in FIG. 2, “baseline-like” aspects of the spectra (low-frequency components and noise), which are mainly related to the blood distance variation, heart motion, and catheter curvature difference, are located in the lowest-frequency approximation component 114G and comprise a majority, approximately 98%, of total spectral variance in many instances. These lowest-frequency components often contain little contribution from the spectral variation caused by the chemical or physical properties of interest.

It is thus possible to establish an operational filter with the available a priori knowledge between analytes of interest and interferants, to maximize the retrieval of the signal of interest from this particular frequency region with a less signal damage and loss, compared with the regular preprocessing methods in single domain.

The subsequently applied regression analysis or discrimination models are either regular single domain methods or dual-domain modeling, according to the invention. The generalized least square (GLS) and orthogonal signal correction have been successfully used as the preprocessing to correct the spectral variation of blood and instrument in single domain. The higher performance of signal correction can be expected when they are applied in dual-domain spectra.

While this invention has been particularly shown and described with references to typical embodiments thereof, it will be understood by those skilled in the art that various changes in form and details may be made therein without departing from the scope of the invention encompassed by the appended claims. Specifically, it is important to note that the use of dual domain techniques described here as pre-processing is independent of the use of dual domain as a chemometric analysis technique. That is, either approaches, or both together can be applied to the spectroscopic data from the vessel walls.

1. A method for optically analyzing blood vessel walls, the method comprising:
receiving optical signals from the vessel walls;
resolving a spectrum of the optical signals to generate spectral data;
transforming the spectral data into dual-domain spectral data;
using the dual-domain spectral data to analyze the vessel walls.

2. A method as claimed in claim 1, wherein the step of transforming the spectral data into dual-domain spectral data comprises applying a wavelet prism.

3. A method as claimed in claim 1, wherein the step of transforming the spectral data into the dual-domain spectral data comprises applying a time-frequency transform and decomposition methods, optimized in response to analytes and interferants.

4. A method as claimed in claim 1, further comprising illuminating the blood vessel walls with an optical source.

5. A method as claimed in claim 4, wherein the optical source generates near infrared light.

6. A method as claimed in claim 1, wherein the step of receiving the optical signals comprises detecting returning radiation to a catheter head.

7. A method as claimed in claim 1, wherein the step of using the dual-domain spectral data to analyze the vessel walls comprises determining whether the blood vessel walls are comprised of vulnerable or non-vulnerable plaques.

8. A method as claimed in claim 1, wherein the step of using the dual-domain spectral data to analyze the vessel walls comprises measuring vulnerability for a risk of heart attack.

9. A method as claimed in claim 1, wherein the step of transforming the spectral data into dual-domain spectral data is performed as a preprocessing step.

10. A method as claimed in claim 1, wherein the step of transforming the spectral data into dual-domain spectral data is performed as a preprocessing step, before application of multivariate regression techniques.

11. A method as claimed in claim 1, wherein the step of transforming the spectral data into dual-domain spectral data is performed as a preprocessing step, before application of a discrimination model.

12. A method as claimed in claim 11, wherein the discrimination model is a single domain model.

13. A method as claimed in claim 11, wherein the discrimination model is a dual domain model.

14. A method as claimed in claim 1, wherein the step of transforming the spectral data into dual-domain spectral data is performed as a preprocessing step that includes removing low-frequency components of the dual-domain spectral data to reduce noise.

15. A method as claimed in claim 1, further comprising preprocessing the spectral data before transforming the spectral data into the dual domain spectral data.

16. A method as claimed in claim 1, wherein the step of using the dual-domain spectral data to analyze the vessel walls comprises applying dual domain multivariate regression techniques.

17. A method as claimed in claim 16, wherein the step of using the dual-domain multivariate regression techniques to analyze the vessel walls comprises applying weight strategy.

18. A method as claimed in claim 17, wherein the step of applying the weight strategy comprises applying cross-validation techniques.

19. A method as claimed in claim 17, wherein the step of applying the weight strategy comprises applying a receiver operating characteristic—area under curve analysis.

20. A method as claimed in claim 1, wherein the step of using the dual-domain spectral data to analyze the vessel walls comprises applying multivariate regression discrimination techniques.

21. A method as claimed in claim 20, wherein the step of using the dual-domain multivariate discrimination techniques to analyze the vessel walls comprises applying a weight strategy.

22. A method as claimed in claim 21, wherein the step of applying the weight strategy comprises applying cross-validation techniques.

23. A method as claimed in claim 21, wherein the step of applying the weight strategy comprises applying the receiver operating characteristic—area under curve analysis.

24. A method as claimed in claim 21, wherein the step of applying the weight strategy comprises applying optimization to maximize separation between discrimination classes and to increase the prediction performance of vulnerability for a risk of heart attack.

25. A method as claimed in claim 20, wherein the step of using the dual-domain multivariate discrimination techniques to analyze the vessel walls comprises applying a receiver operating characteristic—area under curve analysis technique to set a decision boundary.

26. A method as claimed in claim 1, wherein the step of using the dual-domain spectral data to analyze the vessel walls comprises applying a Mahalanobis classifier.

27. A method as claimed in claim 26, wherein the step of applying the dual-domain Mahalanobis classifier comprises applying a receiver operating characteristic—area under curve analysis technique to set decision boundary (surface) in high-dimension space.

28. A system for optically analyzing blood vessel walls, the system comprising:
a detector system for receiving optical signals from the vessel walls;
a spectrometer for resolving a spectrum of the optical signals in wavelength to generate spectral data;
an analyzer for transforming the spectral data into dual-domain spectral data and using the dual-domain spectral data to analyze the vessel walls.

29. A system as claimed in claim 28, wherein the analyzer transforms the spectral data into dual-domain spectral data using a wavelet prism.

30. A system as claimed in claim 28, wherein the analyzer applies a time-frequency transform and decomposition methods, optimized in response to analytes and interferants.

31. A system as claimed in claim 28, further comprising an optical source for illuminating the blood vessel walls.

32. A system as claimed in claim 31, wherein the optical source generates near infrared light.

33. A system as claimed in claim 28, further comprising a catheter head for receiving the optical signals.

34. A system as claimed in claim 28, wherein the analyzer determines whether the blood vessel walls are comprised of vulnerable or non-vulnerable plaques.

35. A system as claimed in claim 28, wherein the analyzer measures a vulnerability for a risk of heart attack.

36. A system as claimed in claim 28, wherein the analyzer transforms the spectral data into dual-domain spectral data to preprocess the spectral data.

37. A system as claimed in claim 28, wherein the analyzer transforms the spectral data into dual-domain spectral data, before applying of multivariate regression techniques.

38. A system as claimed in claim 28, wherein the analyzer transforms the spectral data into dual-domain spectral data, before applying a discrimination model.

39. A system as claimed in claim 38, wherein the discrimination model is a single domain model.

40. A system as claimed in claim 38, wherein the discrimination model is a dual domain model.

41. A system as claimed in claim 28, wherein the analyzer transforms the spectral data into dual-domain spectral data to preprocess the spectral data by removing low-frequency components of the dual-domain spectral data to reduce noise.

42. A system as claimed in claim 28, wherein the analyzer preprocesses the spectral data before transforming the spectral data into the dual domain spectral data.

43. A system as claimed in claim 28, wherein the analyzer applies multivariate regression techniques.

44. A system as claimed in claim 43, wherein the analyzer applies a weight strategy.

45. A system as claimed in claim 44, wherein the application of the weight strategy comprises applying cross-validation techniques.

46. A system as claimed in claim 44, wherein the application of the weight strategy comprises applying a receiver operating characteristic—area under curve analysis.

47. A system as claimed in claim 28, wherein the analyzer applies multivariate regression discrimination techniques.

48. A system as claimed in claim 47, wherein the analyzer applies a weight strategy.

49. A system as claimed in claim 48, wherein the application of the weight strategy comprises applying cross-validation techniques.

50. A system as claimed in claim 48, wherein the application of the weight strategy comprises applying the receiver operating characteristic—area under curve analysis.

51. A system as claimed in claim 47, wherein the analyzer applies a receiver operating characteristic—area under curve analysis technique to set a decision boundary.

52. A system as claimed in claim 28, wherein the analyzer applies Mahalanobis classifier to the dual-domain spectral data to analyze the vessel walls.

53. A system as claimed in claim 52, wherein the analyzer applies a receiver operating characteristic—area under curve analysis technique to set decision boundary (surface) in high-dimension space.
